SEQUENCE LISTING (1 sequence)

SEQ ID NO: 1 - protein, 166 aa, Artificial Sequence
  Amino Acid Sequences Synthesized by Chemical Means
CNLSQTHSLGSKRTLMLLAQMGKISLFSCLKDRHDFEFPQEEFDGNQFQKAQAISVLHELIQQTFNLFSTKESSAAWDEGLLDKFRTELYRQLNDLEACMMQEVGVEETPLMNADSILAVKKYFQRITLYLMEKKYSPCAWEVVRVEIMRSLSFSTNLQKRLRGKD

(19) United States
(12) Patent Application Publication
(10) Pub. No.: US 2023/0088483 A1
(43) Pub. Date: Mar. 23, 2023

(54) USE OF RECOMBINANT CYTOKINE GENE DERIVED PROTEIN OR FRAGMENT THEREOF

(71) Applicants: Genova Inc., Grand Cayman George Town (KY); Genova Biotech (Qingdao) Co., Ltd., Qingdao City, Shandong Province (CN); Beijing Genova Biotech Co., Ltd., Chaoyang District, Beijing (CN)

(72) Inventors: Qiujun LV, Fengtai District, Beijing (CN); Longbin LIU, Chaoyang District, Beijing (CN)

(21) Appl. No.: 17/801,571

(22) PCT Filed: Feb. 24, 2021
(86) PCT No.: PCT/CN2021/077582

§ 371 Date: Aug. 23, 2022

(30) Foreign Application Priority Data
Feb. 24, 2020 (CN) 202010113259.2

(51) Int. Cl. A61K 38/19; A61K 31/56; A61P 31/12

(52) U.S. Cl. CPC A61K 38/19; A61K 31/56; A61P 31/12

(57) ABSTRACT

Provided is a use of a recombinant cytokine gene derived protein or a fragment thereof for inhibiting the activity of a Coronaviridae virus. The recombinant cytokine gene derived protein includes an amino acid sequence represented by SEQ ID NO: 1, or an amino acid sequence that is at least 90% identical to the amino acid sequence represented by SEQ ID NO: 1. Also provided is a use of the described recombinant cytokine gene derived protein or a fragment thereof in the preparation of a drug for preventing or treating a disease or symptom related to the Coronaviridae virus in a subject.

### CROSS-REFERENCE TO RELATED APPLICATIONS

This application is a Section 371 of International Application No. PCT/CN2021/077582, filed Feb. 24, 2021, which was published in the Chinese language on Sep. 2, 2021 under International Publication No. WO 2021/169978 A1, which claims priority under 35 U.S.C. § 119(b) to Chinese Application No. 202010113259.2, filed Feb. 24, 2020, the disclosures of which are incorporated herein by reference in their entirety.

### REFERENCE TO SEQUENCE LISTING SUBMITTED ELECTRONICALLY

This application contains a sequence listing, which is submitted electronically via EFS-Web as an ASCII formatted sequence listing with a file name “689339_29US_Sequence_Listing”, creation date of Aug. 23, 2022, and having a size of 1,773 bytes. The sequence listing submitted via EFS-Web is part of the specification and is herein incorporated by reference in its entirety.

### TECHNICAL FIELD

The present disclosure pertains to the field of antiviral studies, and specifically relates to use of a recombinant cytokine gene derived protein or a fragment thereof for inhibiting activity of Coronaviridae viruses, and to use of the recombinant cytokine gene derived protein or a fragment thereof in preparing a medicine for preventing or treating diseases or symptoms related to the Coronaviridae viruses in subjects.

### BACKGROUND OF THE INVENTION

Coronaviruses are a large family of viruses, and belong to the genus Coronavirus (Coronavirus), the family Coronaviridae (Coronaviridae), and the order Nidovirales (Nidovirales) based on a phylogenetic classification. Viruses of the genus Coronavirus are RNA viruses having envelopes and linear positive-sense single-stranded genomes, and are a large category of viruses that exist widely in nature. Coronaviruses have diameters of approximately 80 nm to 120 nm, methylated cap structures at 5′ ends and poly(A) tails at 3′ ends of their genomes, and genomes of total lengths of approximately 27 kb to 32 kb, which are the largest genomes in currently discovered RNA viruses. However, coronaviruses only infect vertebrates, for example, humans, rats, pigs, cats, dogs, wolves, chickens, cattle, and poultry, are crucial pathogens for many diseases in livestock, pets, and humans, and cause a variety of acute and chronic diseases.

Human coronaviruses was first found in the mid-1960s, and typically cause mild to moderate upper respiratory and gastrointestinal diseases. The 2019-novel coronavirus (2019-nCoV) is a new coronavirus strain, which had not been found in humans until then. 2019-nCoV was identified due to a viral pneumonia case in Wuhan City in 2019, and is the seventh identified coronavirus that can infect humans. 6 other coronaviruses are HCoV-229E, HCoV-OC43, HCoV-NL63, HCoV-HKU1, SARS-CoV (causing severe acute respiratory syndrome), and MERS-CoV (causing Middle East respiratory syndrome). SARS-CoV was first identified in China in 2002 and became a global pandemic in 2002 and 2003; and MERS-CoV was first reported in Saudi Arabia in 2012 and the epidemic spread to several countries. On Feb. 11, 2020, the Coronavirus Study Group (CGS) of the International Committee on Taxonomy of Viruses designated 2019-nCoV as “SARS-CoV-2”. From then on, the 2019-novel coronavirus that caused a large-scale pneumonia pandemic in China has an official name.

The SARS-CoV-2 is extremely contagious, and the main transmission routes are direct transmission, aerosol transmission, and contact transmission. The direct transmission means that infections are caused due to direct inhalation of droplets and exhaled air when a patient is sneezing, coughing, and talking at a close range; the aerosol transmission means that droplets are mixed with the air to form aerosols, and inhalation of the aerosols causes infections; and the contact transmission means that the droplets are deposited on surfaces of objects, hands are contaminated after touching the surfaces, and infections are caused when the hands further touch mucous membranes of the oral cavity, nasal cavity, eyes, and the like. After infection of the SARS-CoV-2, common symptoms of people are respiratory symptoms, fever, cough, polypnea, and dyspnea. In more severe cases, the infection can cause pneumonia, severe acute respiratory syndrome, kidney failure, and even death.

Currently, no specific treatment is available for the disease caused by the SARS-CoV-2, and treatments are provided only as per the symptoms of the patients. Therefore, how to provide a medicine capable of reducing activity of the SARS-CoV-2 and even treating the diseases caused by the SARS-CoV-2 has become a problem that urgently needs to be resolved. The applicant has been dedicated to the research and development of new medicines using biogenic proteins with macromolecular structures for many years, especially the research and development of recombinant cytokine gene derived proteins, and finds that such medicines have good therapeutic effects for tumor diseases and good activity inhibition effects for the chronic hepatitis B virus. In April 2018, the applicant was granted a new drug certificate for a related product “Novaferon” for the treatment of chronic hepatitis B, and the product was put into production. As disclosed in related patent CN200910077177.0 of the applicant, such biogenic proteins are indicated for various viruses such as hepatitis B virus and vesicular stomatitis virus. The applicant unexpectedly finds that the recombinant cytokine gene derived protein independently developed by the applicant has good therapeutic effect on diseases caused by the Coronaviridae viruses, especially the SARS-CoV-2.

### BRIEF SUMMARY OF THE INVENTION

The present disclosure aims to provide a medicine capable of reducing activity of SARS-CoV-2 and even treating a disease caused by the SARS-CoV-2, thereby meeting a current medicine demand for effectively suppressing the SARS-CoV-2. Therefore, after extensive experimental research, the inventor found that a recombinant cytokine gene derived protein or a fragment thereof has an excellent effect of inhibiting the SARS-CoV-2, thereby completing the present disclosure.

According to an aspect, the present disclosure provides use of a recombinant cytokine gene derived protein or a fragment thereof for inhibiting activity of a Coronaviridae virus, where the recombinant cytokine gene derived protein includes an amino acid sequence of SEQ ID NO: 1 or an amino acid sequence having at least 90% identity to the amino acid sequence of SEQ ID NO: 1.

In an embodiment, the polymerase of the Coronaviridae virus is inhibited.

In an embodiment, the Coronaviridae virus may be the SARS-CoV-2, HCoV-229E, HCoV-OC43, HCoV-NL63, HCoV-HKU1, MERS-CoV, or SARS-CoV. In another specific embodiment, the Coronaviridae virus may be the SARS-CoV-2.

In an embodiment, the recombinant cytokine gene derived protein may include an amino acid sequence having at least 93% identity to the amino acid sequence of SEQ ID NO: 1. In another embodiment, the recombinant cytokine gene derived protein may include an amino acid sequence having at least 95% identity to the amino acid sequence of SEQ ID NO: 1. In another embodiment, the recombinant cytokine gene derived protein may include an amino acid sequence having at least 98% identity to the amino acid sequence of SEQ ID NO: 1. In another embodiment, the recombinant cytokine gene derived protein may include an amino acid sequence having at least 99.9% identity to the amino acid sequence of SEQ ID NO: 1.

In an embodiment, the fragment may be a contiguous fragment having more than 50% of a full-length sequence of the recombinant cytokine gene derived protein. In another embodiment, the fragment may be a contiguous fragment having more than 70% of a full-length sequence of the recombinant cytokine gene derived protein. In another embodiment, the fragment may be a contiguous fragment having more than 90% of a full-length sequence of the recombinant cytokine gene derived protein. In another embodiment, the fragment may be a contiguous fragment having more than 99% of a full-length sequence of the recombinant cytokine gene derived protein.

In an embodiment, the recombinant cytokine gene derived protein or its fragment may have enhanced antiviral activity against the Coronaviridae virus compared to human interferon-α2b (HuIFN-α2b). In another embodiment, the recombinant cytokine gene derived protein or its fragment may have anti-viral activity against the Coronaviridae virus at least 2 fold greater than that of HuIFN-α2b. In another embodiment, the recombinant cytokine gene derived protein or its fragment may have anti-viral activity against the Coronaviridae virus at least 5 fold greater than that of HuIFN-α2b. In another embodiment, the recombinant cytokine gene derived protein or its fragment may have anti-viral activity against the Coronaviridae virus at least 10 fold greater than that of HuIFN-α2b. In another embodiment, the recombinant cytokine gene derived protein or its fragment may have anti-viral activity against the Coronaviridae virus at least 50 fold greater than that of HuIFN-α2b.

According to another aspect, the present disclosure further provides use of the recombinant cytokine gene derived protein or a fragment thereof in preparing a medicine for preventing or treating a disease or a symptom related to the Coronaviridae virus in a subject.

In an embodiment, the medicine may include a therapeutically effective dose of recombinant cytokine gene derived protein or its fragment, and a pharmaceutically acceptable excipient, carrier or diluent. In another embodiment, the medicine may further include a therapeutically effective dose of at least one of other antiviral agents or a combination thereof, and the antiviral agent is selected from corticosteroid, an anti-inflammatory signal transduction regulator, a beta-2-adrenergic receptor or agonist or bronchodilator, a PD1 inhibitor, an IL6 inhibitor, an anticholinergic agent, a mucolytic agent, another medicine for treating a coronavirus infection, and a mixture thereof. In another specific embodiment, the other antiviral agents are Remdesivir, Favipiravir, Chloroquine Phosphate, Arbidol, Darunavir, Lopinavir, Interferon, Ribavirin, Lamivudine, Emtricitabine, Tenofovir, Acyclovir, Valacyclovir, Amantadine, Rimantadine, Enfuviride, Maraviroc, Ganciclovir, Saquinavir, Oseltamivir, Zanamivir, Famciclovir, Zidovudine, Efavirenz or Nevirapine, and a mixture thereof.

In an embodiment, the medicine may be prepared as a tablet, a capsule, a pill, a granule, an atomizer, a spray, or an injection.

In an embodiment, the subject may be a human.

In an embodiment, the disease may include at least one of acute respiratory disease, viral pneumonia, severe acute respiratory syndrome, conjunctivitis, and kidney failure. In another embodiment, the symptom may include at least one of nasal congestion, rhinorrhea, fever, cough, sore throat, chest tightness, polypnea, fatigue, anorexia, nausea and vomiting, diarrhea, muscle aches, dyspnea, and hypoxemia.

It is found through research that the recombinant cytokine gene derived protein or its fragment in the present disclosure has an excellent effect of inhibiting activity of the Coronaviridae virus, and therefore has potential to be used as a medicine for preventing or treating the disease or the symptom related to the Coronaviridae virus in the subject. Specifically, compared to a commonly used antiviral agent in the art, that is, human interferon-α2b, the recombinant cytokine gene derived protein or its fragment in the present disclosure may have antiviral activity against the Coronaviridae virus at least 2, 5, 10, or 50 fold greater than that of HuIFN-α2b.

### BRIEF DESCRIPTION OF DRAWINGS

The accompanying drawings are intended to provide a further understanding of the present disclosure and form a part of the specification, and are used together with the following detailed description to explain the present disclosure, but do not constitute a limitation of the present disclosure. In the accompanying drawings:

FIG. 1 shows an inhibitory effect of Novaferon on SARS-CoV-2.

### DETAILED DESCRIPTION OF THE INVENTION

The detailed description of the present disclosure will be described in detail hereafter. It should be understood that, the detailed description described here are only intended to illustrate and explain the present invention, rather than limiting the present invention.

The endpoints and any value of ranges disclosed herein are not limited to precise ranges or values, and these ranges or values should be understood to include values close to these ranges or values. For numerical ranges, combination can be made among the endpoint values of each range, between the endpoint values of each range and individual point values, and among individual point values to obtain one or more new numerical ranges, and these numerical ranges should be regarded as being specifically disclosed herein.

### Definition of Terms

Before detailed description of the present disclosure, it should be understood that the present disclosure is not limited to the specific protein molecules, methods, protocols, cell lines, vectors, and reagents, as these are variable, and it should be further understood that the terms used herein are intended to describe a specific embodiment other than limit the scope of the present invention, where the scope of the present invention is limited only by the appended claims.

To more completely understand the present disclosure described herein, the following terms are used, and the definitions of terms are shown below. Unless otherwise defined, all technical and scientific terms used herein have the same meanings as those understood by persons of ordinary skills in the art to which the present disclosure belongs. All mentioned publications are incorporated herein as reference for the purpose of description and disclosure of the cell lines, vectors, and methods reported in the publications, which may be used in the present disclosure. Nothing herein may be considered as an admission that the present disclosure claims no rights that antedate such disclosures based on prior inventions.

The term “interferon” used herein refers to a family of secreted proteins produced by a variety of eukaryotic cells when exposed to various environmental stimuli (including viral infection or exposure to mitogens), and is a cytokine produced by nuclear cells and lymphocytes. The interferon further has a function of affecting various cells in addition to its antiviral characteristic. Interferon is a broad-spectrum antiviral agent, and does not directly kill or inhibit a virus, but mainly uses a cell surface receptor to stimulate a cell to produce an antiviral protein, thereby inhibiting viral replication. There are mainly three types of interferons: α-interferon (leukocyte), β-interferon (fibroblast), and γ-interferon (lymphocyte). In addition, interferon can also enhance vitality of a natural killer cell (NK cell), macrophage, and a T lymphocyte, to regulate immunity and enhance antiviral ability. Interferons have various biological activities such as broad-spectrum antiviral effects, cell growth effects, and differentiation and immunity regulation functions on the same cells.

The term “human interferon-α2b” or “HuIFN-α2b (Human Interferon-α2b)” used herein is a common one of interferon-α subtype, may be artificially produced through an engineered expression system, and is considered to have functions such as broad-spectrum antiviral ability, and functions of anti-tumor, cell proliferation inhibition, and immunity improvement. For example, interferon is usually used to treat some viral diseases, such as acute and chronic viral hepatitis, herpes zoster, or condyloma acuminatum; or is used to treat some tumors, such as hairy cell leukemia, chronic myeloid leukemia, multiple myeloma, non-Hodgkin's lymphoma, malignant melanoma, renal cell carcinoma, laryngeal papilloma, Kaposi's sarcoma, ovarian cancer, basal cell carcinoma, or bladder cancer.

The term “identity”, “homology”, or “similarity” used herein refers to a similarity percentage of two polynucleotides or parts of a polynucleotide (that is, degree of match with a given amino acid sequence or nucleotide sequence), and may be expressed as a percentage. In the present disclosure, a homologous sequence having the same or similar activity as the given amino acid or nucleotide sequence is described by using “identity percentage”. Identity between one part and another part of a sequence may be determined through techniques known in the art. For example, the identity may be determined by using standard software (specifically, BLAST 2.0) to calculate parameters such as scores, identity, and similarity. Alternatively, sequences are compared by Southern hybridization under strictly defined conditions. Appropriate hybridization conditions can be defined within the art, and the hybridization conditions can be determined in methods (e.g., J. Sambrook et al., Molecular Cloning, A Laboratory Manual, 2nd ed., Cold Spring Harbor Laboratory Press, Cold Spring Harbor, N.Y., 1989; F. M. Ausubel et al., Current Protocols in Molecular Biology, John Wiley & Sons, Inc., New York) well known to persons skilled in the art.

The term “pharmaceutically acceptable” used herein means that a substance (for example, a carrier or diluent) does not affect biological activity or properties of a compound in the present application, and is relatively non-toxic, that is, the substance can be administered to an individual without causing an adverse biological reaction or having interaction in a harmful manner with any constituent contained in the composition. In the present application, the “pharmaceutically acceptable excipient, carrier, or diluent” includes, but is not limited to, any adjuvant, carrier, excipient, glidant, sweetener, diluent, preservative, dye/colorant, flavoring agent, surfactant, wetting agent, dispersant, suspending agent, stabilizer, isotonic agent, solvent, emulsifier, or the like approved for use in humans or livestock by a relevant regulatory authority.

The term “effective dose”, “therapeutically effective dose”, or “pharmaceutically effective dose” used herein refers to a dose of a therapeutic agent that confers a therapeutic effect to a treated subject at an appropriate benefit-risk ratio applicable to any medication. Such a therapeutic effect may be objective (that is, the therapeutic effect may be measured through a specific test or marker) or subjective (that is, the effect is indicated or felt by the subject). In some embodiments, the “therapeutically effective dose” refers to a dose of a therapeutic agent or a composition for effectively treating, improving, or preventing (for example, delaying onset) of a related disease or symptom, and/or conferring a detectable therapeutic or prophylactic effect by improving a symptom associated with a disease, preventing or delaying the onset of the disease, and/or also reducing severity or frequency of the symptom.

The term “treatment” used herein refers to any administration of a therapeutic agent according to a therapeutic regimen, where the therapeutic regimen achieves desired effects, that is, effects of partially or completely relieving, improving, remitting, inhibiting, delaying, and reducing severity and/or incidence of one or more symptoms or conditions of a particular disease, disorder, and/or condition (for example, chronic or recurrent immune responses and gastrointestinal (GI) inflammation, or chronic or recurrent hyperglycemia); and in some embodiments, administration of a therapeutic agent according to a therapeutic regimen is associated with achievement of desired effects. Such treatment may be prescribed for a subject who do not have a relevant disease, disorder, and/or symptom, and/or a subject having only an early sign of the disease, disorder and/or condition. Alternatively or additionally, such treatment may be prescribed for a subject having one or more established signs of the relevant disease, disorder, and/or condition. In some embodiments, the treatment may be prescribed for a subject who has been diagnosed with the relevant disease, disorder, and/or condition. In some embodiments, the treatment may be prescribed for a subject for whom one or more predisposing factors have been identified, where the predisposing factors are statistically associated with an increased risk of developing the relevant disease, disorder, and/or condition.

The terms “subject”, “patient”, and “individual” used herein refer to a human or non-human animal. These terms include mammals, such as, humans, non-human primates, livestock (for example, cattle, pigs, sheep, goats, and poultry), companion animals (for example, dogs, cats, horses, and rabbits), and rodents (for example, mice and rats). In some embodiments, the term refers to a human subject. In an exemplary embodiment, the term refers to a human subject suffering from a disease or a symptom associated with, for example, Coronaviridae viruses or any combination thereof.

The term “medicine” used herein includes a medicine used for humans and animals in human and veterinary medicine. Additionally, the term “medicine” used herein refers to any substance that provides a therapeutic and/or beneficial effect. The term “medicine” used herein is not necessarily limited to substances requiring approval for marketing, but may include substances that can be used in cosmetics, nutraceuticals, foods (including, for example, feed and beverages), probiotic cultures, nutritional supplements, and natural remedies. Additionally, the term “medicine” used herein includes products designed to be doped into animal feed (for example, livestock feed and/or pet food).

### Recombinant Cytokine Gene Derived Protein

The recombinant cytokine gene derived protein is an analog of interferon, and is usually artificially synthesized through a recombinant technology, instead of naturally occurring. For example, a host cell may be transfected with a recombinant vector including a DNA molecule encoding the recombinant cytokine gene derived protein, to obtain the recombinant cytokine gene derived protein or its fragment. The recombinant vector can be, for example, a plasmid, phage, or a viral or retroviral vector. The retroviral vector can be replication-competent or replication-deficient. In a latter case, viral propagation usually occurs only in complementary host cells.

According to an aspect, the present disclosure provides use of a recombinant cytokine gene derived protein or its fragment for inhibiting activity of a Coronaviridae virus, where the recombinant cytokine gene derived protein includes an amino acid sequence of SEQ ID NO: 1 or an amino acid sequence having at least 90% identity to the amino acid sequence of SEQ ID NO: 1.

The inventor of the present disclosure finds through research that the recombinant cytokine gene derived protein or its fragment in the present disclosure can at least inhibit polymerase of the Coronaviridae virus, to inhibit the activity of the Coronaviridae virus. Therefore, in some embodiments, the polymerase of the Coronaviridae virus may be inhibited. In addition, the Coronaviridae virus may include all currently known Coronaviridae viruses, and similar viruses that are to be further discovered in the future and that belong to the family Coronaviridae. Currently, the Coronaviridae virus may specifically include a Coronaviridae virus known to infect humans. For example, the Coronaviridae virus may be SARS-CoV-2, HCoV-229E, HCoV-OC43, HCoV-NL63, HCoV-HKU1, MERS-CoV, or SARS-CoV. In another embodiment, the Coronaviridae virus may specifically further be the SARS-CoV-2.

Although a protein with anti-viral activity against the Coronaviridae virus in the present disclosure can be directly a protein including an amino acid sequence of SEQ ID NO: 1, the protein does not exclude a sequence added upstream or downstream of the amino acid sequence of SEQ ID NO: 1, and naturally occurring mutations or silent mutations. It is obvious to persons skilled in the art that when a protein has the same or similar activity as the protein including the amino acid sequence of SEQ ID NO: 1, the protein belongs to the protein with the anti-viral activity against the Coronaviridae virus in the present disclosure. Therefore, the protein with the anti-viral activity against the Coronaviridae virus in the present disclosure may be a protein including the amino acid sequence of SEQ ID NO: 1 or an amino acid sequence having at least 90% identity to the amino acid sequence of SEQ ID NO: 1. In addition, it is obvious that any protein having an amino acid sequence a part of which is deleted, modified, substituted or added may also fall within the scope of the present disclosure, provided that the protein has any of the foregoing amino acid sequences with identity and has a function corresponding to the foregoing protein.

Therefore, in an embodiment, the recombinant cytokine gene derived protein may include an amino acid sequence having at least 91% identity to the amino acid sequence of SEQ ID NO: 1. In another embodiment, the recombinant cytokine gene derived protein may include an amino acid sequence having at least 92% identity to the amino acid sequence of SEQ ID NO: 1. In another embodiment, the recombinant cytokine gene derived protein may include an amino acid sequence having at least 93% identity to the amino acid sequence of SEQ ID NO: 1. In another embodiment, the recombinant cytokine gene derived protein may include an amino acid sequence having at least 94% identity to the amino acid sequence of SEQ ID NO: 1. In another embodiment, the recombinant cytokine gene derived protein may include an amino acid sequence having at least 95% identity to the amino acid sequence of SEQ ID NO: 1. In another embodiment, the recombinant cytokine gene derived protein may include an amino acid sequence having at least 96% identity to the amino acid sequence of SEQ ID NO: 1. In another embodiment, the recombinant cytokine gene derived protein may include an amino acid sequence having at least 97% identity to the amino acid sequence of SEQ ID NO: 1. In another embodiment, the recombinant cytokine gene derived protein may include an amino acid sequence having at least 98% identity to the amino acid sequence of SEQ ID NO: 1. In another embodiment, the recombinant cytokine gene derived protein may include an amino acid sequence having at least 99% identity to the amino acid sequence of SEQ ID NO: 1. In another embodiment, the recombinant cytokine gene derived protein may include an amino acid sequence having at least 99.9% identity to the amino acid sequence of SEQ ID NO: 1.

In addition, the recombinant cytokine gene derived protein in the present disclosure may exist in its fragment form (or referred to as “its section form”), provided that the fragment can also have the function corresponding to the foregoing proteins. Therefore, in an embodiment, the fragment may be a partial contiguous fragment of a full-length sequence of the recombinant cytokine gene derived protein, without affecting the function of the foregoing protein. In another embodiment, the fragment is a contiguous fragment having more than 50% of a full-length sequence of the recombinant cytokine gene derived protein. In another embodiment, the fragment is a contiguous fragment having more than 60% of a full-length sequence of the recombinant cytokine gene derived protein. In another embodiment, the fragment may be a contiguous fragment having more than 70% of a full-length sequence of the recombinant cytokine gene derived protein. In another embodiment, the fragment is a contiguous fragment having more than 80% of a full-length sequence of the recombinant cytokine gene derived protein. In another embodiment, the fragment may be a contiguous fragment having more than 90% of a full-length sequence of the recombinant cytokine gene derived protein. In another embodiment, the fragment may be a contiguous fragment having more than 99% of a full-length sequence of the recombinant cytokine gene derived protein.

As mentioned above, the recombinant cytokine gene derived protein or its fragment in the present disclosure has an excellent effect of inhibiting the SARS-CoV-2, and may be used for medicine for reducing the activity of the SARS-CoV-2 and even treating the disease caused by the SARS-CoV-2. In the present disclosure, the inventor compares the anti-viral activity against the Coronaviridae virus of the recombinant cytokine gene derived protein or its fragment with human interferon-α2b (HuIFN-α2b), a common broad-spectrum antiviral agent in the art, and then finds that the recombinant cytokine gene derived protein or its fragment has enhanced anti-viral activity against the Coronaviridae virus compared to human interferon-α2b (HuIFN-α2b). In an embodiment, the recombinant cytokine gene derived protein or its fragment may have anti-viral activity against the Coronaviridae virus at least 2 fold greater than that of HuIFN-α2b. In another embodiment, the recombinant cytokine gene derived protein or its fragment may have anti-viral activity against the Coronaviridae virus at least 5 fold greater than that of HuIFN-α2b. In another embodiment, the recombinant cytokine gene derived protein or its fragment may have anti-viral activity against the Coronaviridae virus at least 10 fold greater than that of HuIFN-α2b. In another embodiment, the recombinant cytokine gene derived protein or its fragment may have anti-viral activity against the Coronaviridae virus at least 20 fold greater than that of HuIFN-α2b. In another embodiment, the recombinant cytokine gene derived protein or its fragment may have anti-viral activity against the Coronaviridae virus at least 50 fold greater than that of HuIFN-α2b. In another embodiment, the recombinant cytokine gene derived protein or its fragment may have anti-viral activity against the Coronaviridae virus at least 100 fold greater than that of HuIFN-α2b. In another embodiment, the recombinant cytokine gene derived protein or its fragment may have anti-viral activity against the Coronaviridae virus at least 200 fold greater than that of HuIFN-α2b.

### Pharmaceutical Forms and Administration

According to another aspect, the present disclosure further provides use of the recombinant cytokine gene derived protein or its fragment in preparing a medicine for preventing or treating a disease or a symptom related to the Coronaviridae virus in a subject. In an embodiment, the medicine may include a therapeutically effective dose of recombinant cytokine gene derived protein or its fragment, and a pharmaceutically acceptable excipient, carrier or diluent.

In the present disclosure, the subject or patient to which the medicine is to be administered may be a human or a non-human animal. Specifically, these terms may include mammals, such as, humans, non-human primates, livestock (for example, cattle, pigs, sheep, goats, and poultry), companion animals (for example, dogs, cats, horses, and rabbits), and rodents (for example, mice and rats). In some embodiments, the subject is a human subject.

Persons skilled in the art should understand that the therapeutically effective dose of the to-be-administered recombinant cytokine gene derived protein or its fragment depends on the following items: nature and severity of the subject and disease, a physical condition of the subject, a treatment regimen (for example, with or without a second therapeutic agent), and a selected route of administration; and an appropriate dose can be readily determined by persons skilled in the art. Additionally, the optimal dose and interval of the medicine for individuals depends on nature and severity of a treated condition, a form, a route, and a location of administration, and age and condition of a specific treated subject, and an appropriate to-be-administered dose is finally at the discretion of the physician. The dose can be repeatedly used multiple times as needed. If a side effect occurs, the dose and/or frequency can be changed or reduced based on normal clinical practice. In an embodiment, the medicine may conveniently be presented in a unit dosage form, so that each dose contains a predetermined amount of recombinant cytokine gene derived protein or its fragment. Such unit dosage may contain 0.5 mg to 5 g, including but not limited to, for example, 1 mg, 10 mg, 20 mg, 30 mg, 40 mg, 50 mg, 100 mg, 200 mg, 300 mg, 400 mg, 500 mg, 750 mg, or 1000 mg, or any range between any two of the foregoing values, for example, 10 mg to 1000 mg, 20 mg to 50 mg, or 30 mg to 300 mg.

The treatment regimen can vary from monthly to daily based on various clinical factors, including a disease type, disease severity, and sensitivity of a patient. In a specific embodiment, the medicine is administered once daily, twice a week, three times a week, once every other day, once every 5 days, once a week, once every 10 days, once every two weeks, once every three weeks, once every four weeks, or once a month, or is administered within any range between any two of the foregoing values, for example, once every four days to once a month, once every 10 days to once every two weeks, two to three times a week, or the like.

In addition, a wide range of forms of pharmaceutically acceptable excipients, carriers, or diluents can be used based, for example, on the to-be-treated condition or the route of administration. In the present disclosure, there are a variety of pharmaceutically acceptable excipients, carriers, or diluents, including but not limited to, for example, buffers, stabilizers, isotonic agents, nonionic detergents, antioxidants, and various other additives.

The buffer helps maintain pH within a range close to physiological conditions. The buffer can be at a concentration ranging from about 2 mM to about 50 mM. Suitable buffers include organic and inorganic acids and their salts, for example, a citrate buffer, a citrate-phosphate buffer, a succinate buffer, a tartrate buffer, a fumarate buffer, a gluconic acid salt buffer, an oxalate buffer, a lactate buffer, and an acetate buffer. In addition, the phosphate buffer, the histidine buffer, and trimethylamine salt such as Tris can be used. The isotonic agents can ensure isotonicity of the liquid composition and include polyhydroxy sugar alcohol, or preferably, trihydroxy sugar alcohol or a sugar alcohol of a higher grade, for example, glycerol, erythritol, arabitol, xylose alcohol, sorbitol, and mannitol. The stabilizers refer to a broad range of excipients having such functions as bulking agents and additives for dissolving the therapeutic agent, helping prevent degeneration, or adhering to a container wall. Typical stabilizers can be polyhydroxy sugar alcohol, amino acids, organic sugar or sugar alcohol, cyclic polyol, amino acid polymer, sulfur-containing reductant, low molecular weight polypeptide (for example, peptide having 10 or less residues), protein, hydrophilic polymer, disaccharide, polysaccharide, and the like. The stabilizer may be in a range of 0.1 to 10,000 parts by weight of active protein. The nonionic surfactant or detergent (also referred to as “wetting agent”) can help dissolve the therapeutic agent and protect the therapeutic protein from agitation-induced aggregation, so that the formulation can be exposed to shear surface stress without causing denaturation of the protein. The suitable nonionic surfactant includes polysorbate (20, 80, and the like), poloxamer (184, 188, and the like), Pluronic polyol, polyoxyethylene sorbitan monoether (TWEEN®-20, TWEEN®-80, and the like). The nonionic surfactant may be at a concentration ranging from about 0.05 mg/mL to about 1.0 mg/mL, or from about 0.07 mg/mL to about 0.2 mg/mL. Various other additives may also include a bulking agent (for example, starch), a chelating agent (for example, EDTA), antioxidant (for example, ascorbic acid, methionine, vitamin E), a protease inhibitor, a co-solvent, and the like.

According to the present disclosure, the recombinant cytokine gene derived protein or its fragment in the present disclosure can also be optionally co-administered with one or more other antiviral agents to achieve a higher therapeutic effect. Therefore, in an embodiment, the medicine may further include a therapeutically effective dose of at least one of other antiviral agents or a combination thereof, and the antiviral agent is selected from corticosteroid, an anti-inflammatory signal transduction regulator, a beta-2-adrenergic receptor or agonist or bronchodilator, a PD1 inhibitor, an IL6 inhibitor, an anticholinergic agent, a mucolytic agent, another medicine for treating a coronavirus infection, and a mixture thereof. In a more specific embodiment, the other antiviral agents may be Remdesivir, Favipiravir, Chloroquine Phosphate, Arbidol, Darunavir, Lopinavir, Interferon, Ribavirin, Lamivudine, Emtricitabine, Tenofovir, Acyclovir, Valacyclovir, Amantadine, Rimantadine, Enfuviride, Maraviroc, Ganciclovir, Saquinavir, Oseltamivir, Zanamivir, Famciclovir, Zidovudine, Efavirenz or Nevirapine, and a mixture thereof. When the medicine contains a plurality of effective constituents, the effective constituents may be administered simultaneously, sequentially, or separately at the discretion of the physician.

In addition, the recombinant cytokine gene derived protein or its fragment in the present disclosure can be administered to a patient by a variety of routes, for example, the oral, transdermal, subcutaneous, intranasal, intravenous, intramuscular, intrathecal, regional, or topical (for example, mucosa) route. The most appropriate route of administration in any given situation depends on the subject and the nature and severity of the disease, physical conditions of the subject, and the like. In an embodiment, the recombinant cytokine gene derived protein or its fragment in the present disclosure may be administered intravenously. In another embodiment, the recombinant cytokine gene derived protein or its fragment in the present disclosure may be administered orally. Correspondingly, based on different routes of administration, the medicine in the present disclosure can be prepared into different dosage forms. For example, in an embodiment, the medicine may be prepared as a tablet, a capsule, a pill, a granule, an atomizer, a spray, or an injection. In a preferable embodiment, the medicine is prepared as the spray. In a more preferable embodiment, the medicine is Novaferon (Novaferon).

### To-Be-Treated Symptom

Based on a mechanism of action of the recombinant cytokine gene derived protein or its fragment in the present disclosure, most diseases or symptoms associated with Coronaviridae viruses can be prevented or treated. Specifically, in an embodiment, the disease may include at least one of acute respiratory disease, viral pneumonia, severe acute respiratory syndrome, conjunctivitis, and kidney failure. In another embodiment, the symptom may include at least one of nasal congestion, rhinorrhea, fever, cough, sore throat, chest tightness, polypnea, fatigue, anorexia, nausea and vomiting, diarrhea, muscle aches, dyspnea, and hypoxemia.

It is found through research that the medicine prepared from the recombinant cytokine gene derived protein or its fragment in the present disclosure has great potential in preventing or treating diseases or symptoms related to Coronaviridae viruses. Specifically, compared to a commonly used antiviral agent in the art, that is, human interferon-α2b, the recombinant cytokine gene derived protein or its fragment in the present disclosure may have a prophylactic or therapeutic effect at least 2, 5, 10, or 50 fold greater than that of HuIFN-α2b.

The following example describes the anti-viral activity against the Coronaviridae virus of the recombinant cytokine gene derived protein or its fragment in the present disclosure in detail.

### Example

In this example, Novaferon (Novaferon, Genova Biotech (Qingdao) Co., Ltd.) was tested for inhibition of the SARS-CoV-2 in vitro by using the 96-well plate method. Details are as follows:

Vero cells were selected as a cell strain, and inoculated into the plate at a cell concentration of 10000 cells/well (100 μL) to form a monolayer for 24 hours, and then the SARS-CoV-2 (C-Tan-nCoV Wuhan strain 01) was selected as a virus strain, and inoculated into each well at a concentration of 100 PFU/well. 2 hours after the cells were infected by the virus, Novaferon was added to each well at different concentrations (concentrations were 0.001 ng/mL, 0.01 ng/mL, 0.1 ng/mL, 1 ng/mL, 10 ng/mL, and 100 ng/mL, respectively), the wells were used as a treatment group, and wells without Novaferon were used as a control group.

24 hours after the cells were treated with the medicine at different concentrations, 100 μL of cell supernatant was extracted from each well respectively to extract nucleic acids (automated nucleic acid extraction instrument, Suzhou Tianlong Biotechnology Co., Ltd.), and the extracted nucleic acids were subjected to RT-PCR (TaKaRa) and qRT-PCR tests (Roche 480, target ORF1a/b, forward primer (F): CCCTGTGGGTTTTACACTTAA; reverse primer (R): ACGATTGTGCATCAGCTGA; and fluorescent probe (P): 5′-FAM-CCGTCTGCG . . . -BHQ1-3′) to measure Ct values of the viruses with the existence of the medicine at different concentrations. See Table 1 for the results.

TABLE 1
Inhibitory Effect of Novaferon on SARS-CoV-2.

| Concentration of | |
|---|---|
| medicine (ng/mL) | Ct value |
| 100 | 27.625 |
| 10 | 29.025 |
| 1 | 23.665 |
| 0.1 | 22.815 |
| 0.01 | 22.685 |
| 0.001 | 22.645 |
| Control group | 22.6 |

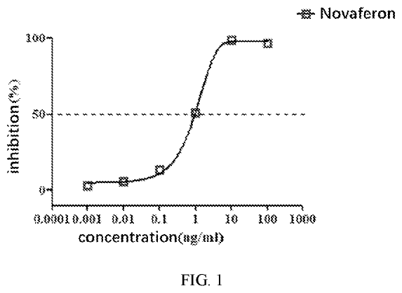

Then inhibition rates of medicine at different concentrations for the viruses were calculated and plotted based on the Ct values of the viruses in Table 1. See FIG. 1 for the results. Preliminary calculations showed that half maximal inhibitory concentration IC50 of Novaferon for the SARS-CoV-2 in vitro was 1.02 ng/mL.

It could be seen from the results that the medicine of the recombinant cytokine gene derived protein (for example, Novaferon) in the present disclosure actually had an effective inhibitory effect on a cytopathic effect of the SARS-CoV-2 in vitro, and had an IC50 value of 1.02 ng/mL, and when the concentration of the medicine in the present disclosure reached 10 ng/mL and 100 ng/mL, no obvious cytopathic effect (CPE) was observed.

In addition, because an atomization concentration of Novaferon was 20 μg/mL, approximately equivalent to 20000 times its half maximal inhibitory concentration, an effective concentration of Novaferon can be ensured in the respiratory tract after atomization inhalation, so as to inhibit reproduction of the coronaviruses.

The preferred embodiments of the present invention have been described in detail above. However, the present invention is not limited to the specific details in the above embodiments. Within the scope of the technical concept of the present invention, many simple modifications can be made to the technical solution of the present invention, all of which fall within the claimed scope of the present invention.

Furthermore, it should be noted that various specific technical features described in the above specific embodiments can be combined in any suitable way without contradiction. In order to avoid unnecessary repetition, the present invention will not explain various possible combinations separately.

Furthermore, any combination can be made among various embodiments of the present invention, as long as it does not violate the idea of the present invention, it should also be regarded as the disclosure of the present invention.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

1. Use of a recombinant cytokine gene derived protein or a fragment thereof for inhibiting activity of a Coronaviridae virus, wherein the recombinant cytokine gene derived protein comprises an amino acid sequence of SEQ ID NO: 1, or an amino acid sequence having at least 90% identity to the amino acid sequence of SEQ ID NO: 1.

2. The use according to claim 1, wherein polymerase of the Coronaviridae virus is inhibited.

3. The use according to claim 1, wherein Coronaviridae virus is SARS-CoV-2, HCoV-229E, HCoV-OC43, HCoV-NL63, HCoV-HKU1, MERS-CoV, or SARS-CoV.

4. The use according to claim 3, wherein the Coronaviridae virus is the SARS-CoV-2.

5. The use according to claim 1, wherein the recombinant cytokine gene derived protein comprises an amino acid sequence having at least 93% identity to the amino acid sequence of SEQ ID NO: 1.

6. The use according to claim 5, wherein the recombinant cytokine gene derived protein comprises an amino acid sequence having at least 95% identity to the amino acid sequence of SEQ ID NO: 1.

7. The use according to claim 6, wherein the recombinant cytokine gene derived protein comprises an amino acid sequence having at least 98% identity to the amino acid sequence of SEQ ID NO: 1.

8. The use according to claim 7, wherein the recombinant cytokine gene derived protein comprises an amino acid sequence having at least 99.9% identity to the amino acid sequence of SEQ ID NO: 1.

9. The use according to claim 1, wherein the fragment is a contiguous fragment having more than 50% of a full-length sequence of the recombinant cytokine gene derived protein.

10. The use according to claim 9, wherein the fragment is a contiguous fragment having more than 70% of a full-length sequence of the recombinant cytokine gene derived protein.

11. The use according to claim 10, wherein the fragment is a contiguous fragment having more than 90% of a full-length sequence of the recombinant cytokine gene derived protein.

12. The use according to claim 11, wherein the fragment is a contiguous fragment having more than 99% of a full-length sequence of the recombinant cytokine gene derived protein.

13. The use according to claim 1, wherein the recombinant cytokine gene derived protein or the fragment thereof has enhanced antiviral activity against the Coronaviridae virus compared to human interferon-α2b (HuIFN-α2b).

14. The use according to claim 13, wherein the recombinant cytokine gene derived protein or the fragment thereof has anti-viral activity against the Coronaviridae virus at least 2 fold greater than that of HuIFN-α2b.

15. The use according to claim 14, wherein the recombinant cytokine gene derived protein or the fragment thereof has anti-viral activity against the Coronaviridae virus at least 5 fold greater than that of HuIFN-α2b.

16. The use according to claim 15, wherein the recombinant cytokine gene derived protein or the fragment thereof has anti-viral activity against the Coronaviridae virus at least 10 fold greater than that of HuIFN-α2b.

17. The use according to claim 16, wherein the recombinant cytokine gene derived protein or the fragment thereof has anti-viral activity against the Coronaviridae virus at least 50 fold greater than that of HuIFN-α2b.

18. Use of the recombinant cytokine gene derived protein or the fragment thereof according to claim 1 in preparing a medicine for preventing or treating a disease or a symptom related to the Coronaviridae virus in a subject.

19. The use according to claim 18, wherein the medicine comprises a therapeutically effective dose of recombinant cytokine gene derived protein or the fragment thereof, and a pharmaceutically acceptable excipient, carrier, or diluent.

20. The use according to claim 19, wherein the medicine further comprises a therapeutically effective dose of at least one of other antiviral agents or a combination thereof, and the antiviral agent is selected from corticosteroid, an anti-inflammatory signal transduction regulator, a beta-2-adrenergic receptor or agonist or bronchodilator, a PD1 inhibitor, an IL6 inhibitor, an anticholinergic agent, a mucolytic agent, another medicine for treating a coronavirus infection, and a mixture thereof.

21. The use according to claim 20, wherein the other antiviral agents are Remdesivir, Favipiravir, Chloroquine Phosphate, Arbidol, Darunavir, Lopinavir, Interferon, Ribavirin, Lamivudine, Emtricitabine, Tenofovir, Acyclovir, Valacyclovir, Amantadine, Rimantadine, Enfuviride, Maraviroc, Ganciclovir, Saquinavir, Oseltamivir, Zanamivir, Famciclovir, Zidovudine, Efavirenz or Nevirapine, and a mixture thereof.

22. The use according to claim 18, wherein the medicine is prepared as a tablet, a capsule, a pill, a granule, an atomizer, a spray, or an injection.

23. The use according to claim 18, wherein the subject is a human.

24. The use according to claim 18, wherein the disease comprises at least one of acute respiratory disease, viral pneumonia, severe acute respiratory syndrome, conjunctivitis, and kidney failure.

25. The use according to claim 18, wherein the symptom comprises at least one of nasal congestion, rhinorrhea, fever, cough, sore throat, chest tightness, polypnea, fatigue, anorexia, nausea and vomiting, diarrhea, muscle aches, dyspnea, and hypoxemia.
